(19) United States
(12) Patent Application Publication
(10) Pub. No.: US 2024/0327887 A1
(43) Pub. Date: Oct. 3, 2024

(54) SLOWLY-DIGESTED PUERARIA MONTANA VAR. THOMSONII STARCH AND PREPARATION METHOD THEREOF

(71) Applicant: JIANGXI UNIVERSITY OF CHINESE MEDICINE, Nanchang (CN)

(72) Inventors: Huanhuan DONG, Nanchang (CN); Xuantong DUAN, Nanchang (CN); Yongmei GUAN, Nanchang (CN); Weifeng ZHU, Nanchang (CN); Lihua CHEN, Nanchang (CN); Lijun PENG, Nanchang (CN); Ying ZHOU, Nanchang (CN)

(73) Assignee: JIANGXI UNIVERSITY OF CHINESE MEDICINE, Nanchang (CN)

(21) Appl. No.: 18/575,307

(22) PCT Filed: Dec. 23, 2022
(86) PCT No.: PCT/CN2022/141261

§ 371 Date: Dec. 29, 2023

(30) Foreign Application Priority Data
Dec. 28, 2021 (CN) 202111620836.8

(51) Int. Cl. C12P 19/04; A61K 31/718; C12P 19/14

(52) U.S. Cl. CPC C12P 19/04; A61K 31/718; C12P 19/14

(57) ABSTRACT

A preparation method of a slowly-digested Pueraria montana var. thomsonii starch is provided. The preparation method includes: cleaning, peeling, and chopping Pueraria montana var. thomsonii, slurrying, subjecting a resulting slurry to coarse filtration, and allowing a resulting filtrate to fully stand; collecting a supernatant obtained after the standing, and adding α-amylase to allow enzymatic hydrolysis; inactivating the enzyme, concentrating, centrifuging, and discarding a resulting precipitate to obtain a first Pueraria montana var. thomsonii concentrate composition; collecting a precipitate obtained after the standing, repeatedly cleaning and settling the precipitate 2 to 3 times to obtain a second Pueraria montana var. thomsonii starch composition; mixing the first Pueraria montana var. thomsonii concentrate composition and the second Pueraria montana var. thomsonii starch composition, and adding deionized water for dilution to obtain a homogeneous slurry; and drying and crushing the homogeneous slurry, and sieving a crushed material.

### CROSS REFERENCE TO THE RELATED APPLICATIONS

This application is the national phase entry of International Application No. PCT/CN2022/141261, filed on Dec. 23, 2022, which is based upon and claims priority to Chinese Patent Application No. 202111620836.8, filed on Dec. 28, 2021, the entire contents of which are incorporated herein by reference.

### TECHNICAL FIELD

The present disclosure belongs to the technical field of food processing, and specifically relates to a slowly-digested Pueraria montana var. thomsonii starch and a preparation method thereof.

### BACKGROUND

The dietary structure of people is slowly changing with the fast-paced life, and some bad eating habits of modern people also lead to constant changes in the dietary structure. The refinement of diets and the reduction of exercise leads to metabolism-associated chronic diseases such as diabetes, obesity, and cardiovascular diseases (CVDs), which pose a potential threat to human health. A scientific and reasonable dietary structure and active and effective dietary intervention are effective ways to prevent and control the above diseases. Therefore, it is very necessary to actively prevent and treat diabetes, and the establishment of a correct diet concept and the adoption of a reasonable lifestyle can minimize the incidence rate of diabetes. For either type 1 diabetes or type 2 diabetes, a low-glycemic index (GI) diet therapy is the cornerstone of diabetes treatment. A large number of studies have shown that low-GI foods play a prominent role in maintaining physical health and lowering blood glucose level. A low-GI diet can reduce the blood glucose level and maintain a steady blood glucose level in diabetes patients, and also plays an indispensable role in weight loss and blood lipid level lowering. For a gestational diabetes population, a low-GI diet can also reduce the risk of pregnancy complications, and thus has gradually become a mainstream dietary intervention strategy for diabetes patients.

Starch is a main source of energy for the human body, and according to the release rate of glucose after starch intake, starch can include rapidly-digested starch (RDS), slowly-digested starch (SDS), and resistant starch (RS). RDS can be completely hydrolyzed and absorbed in the digestive tract at a high digestion rate, and can cause a rapid rise in a blood glucose level. SDS is absorbed in the small intestine at a relatively-low rate, and provides energy through the continuous and slow release of glucose. SDS can reduce the rate of postprandial insulin secretion and maintain a steady state of blood glucose, and is suitable for diabetes patients. RS cannot be absorbed and is decomposed and utilized by intestinal flora in the large intestine, which is similar to dietary fibers. Pueraria montana var. thomsonii was originally recorded in the “Shen Nong's Herbal Classic”, and belongs to the Pueraria genus of the Fabaceae family. There is a long history of folk consumption of Pueraria montana var. thomsonii, and Pueraria montana var. thomsonii is known as “Asian ginseng”. Pueraria montana var. thomsonii is a traditional medicinal and edible plant, is rich in a variety of nutrients, and has been listed by the Food and Agriculture Organization (FAO) as one of the six major grain crops. Pueraria montana var. thomsonii starch has a high nutritional value and a starch content of 60% or more, is easy to consume, and is deeply favored by consumers. However, Pueraria montana var. thomsonii starch is mainly RDS, and may cause a rapid rise in the blood glucose level after being consumed by the human body. Therefore, Pueraria montana var. thomsonii starch is not suitable for people with high blood glucose levels. The increase of SDS and RS contents in starch by modification has become a research hotspot.

In previous studies, it has been found that the RDS content in Pueraria montana var. thomsonii starch is relatively high. Therefore, it is necessary to develop a slowly-digested Pueraria montana var. thomsonii starch.

### SUMMARY

In view of the above problems existing in the prior art, an objective of the present disclosure is to provide a slowly-digested Pueraria montana var. thomsonii starch with SDS and RS contents improved, and a preparation method thereof.

The objective of the present disclosure is achieved by the following technical solutions:

The present disclosure provides a preparation method of a slowly-digested Pueraria montana var. thomsonii starch, including the following steps:
(1) taking fresh Pueraria montana var. thomsonii, a medicinal and edible plant, as a raw material, and cleaning, peeling, and chopping the raw material; and adding 52° C. to 55° C. hot water at a weight 5 to 8 times a weight of the raw material, fully crushing, sieving to remove crude fibers, and allowing a resulting slurry to stand;
(2) collecting a supernatant obtained after the standing in the step (1), heating the supernatant to 52° C. to 55° C., adding α-amylase according to a weight ratio of 0.1% to 0.6%, and stirring a resulting mixture to allow enzymatic hydrolysis for 1 h to 3 h; heating a resulting reaction system to 95° C. and holding the temperature for 5 min to 10 min to inactivate the enzyme, and reducing a pressure of an inactivated system obtained after the enzyme inactivation to 0.1 KPa to 0.15 KPa; and concentrating and centrifuging a resulting solution, and discarding a resulting precipitate to obtain a first Pueraria montana var. thomsonii concentrate composition;
(3) collecting a precipitate obtained after the standing in the step (1), repeatedly cleaning and settling the precipitate with drinking water 2 to 3 times, and collecting a final precipitate to obtain a second Pueraria montana var. thomsonii starch composition;
(4) mixing the first Pueraria montana var. thomsonii concentrate composition obtained in the step (2) and the second Pueraria montana var. thomsonii starch composition obtained in the step (3) according to a weight ratio of (0.5-5):100, and adding 40 to 60 parts of deionized water for dilution to obtain a homogeneous slurry; and
(5) drying and crushing the homogeneous slurry obtained in the step (4), and sieving a crushed material through an 80-mesh sieve to obtain the slowly-digested Pueraria montana var. thomsonii starch.

Further, the slowly-digested Pueraria montana var. thomsonii starch includes 10% to 25% of SDS and 8% to 20% of RS.

Preferably, in the step (4), the first Pueraria montana var. thomsonii concentrate composition and the second Pueraria montana var. thomsonii starch composition are mixed according to a weight ratio of 5:100.

The present disclosure also provides a use of the slowly-digested Pueraria montana var. thomsonii starch as a major raw material in development and production of a low-GI cooked wheaten food, pastry, and solid beverage.

The present disclosure also provides a use of the slowly-digested Pueraria montana var. thomsonii starch in preparation of a product for non-therapeutic intervention of a blood glucose level.

Further, the non-therapeutic intervention of a blood glucose level is specifically manifested as at least one selected from the group consisting of the following conditions (a) to (d):
(a) regulating the blood glucose level;
(b) extending a time required to reach a blood glucose peak;
(c) lowering the blood glucose level and improving glucose tolerance; and
(d) improving a pancreatic islet function.

The slowly-digested Pueraria montana var. thomsonii starch of the present disclosure has the following characteristics and beneficial effects:
1. In the present disclosure, with a Pueraria montana var. thomsonii starch as a raw material, a slowly-digested Pueraria montana var. thomsonii starch is prepared; and the slowly-digested Pueraria montana var. thomsonii starch includes 10% to 25% of SDS and 8% to 20% of RS.
2. The preparation method of the present disclosure can increase an SDS content in a Pueraria montana var. thomsonii starch by 10% or more and prolong a digestion time of the starch by 45.5%, and the prepared slowly-digested Pueraria montana var. thomsonii starch can reduce a GI, regulate a blood glucose response, prolong a time required to reach a blood glucose peak, reduce a blood glucose level, improve the glucose tolerance and pancreatic islet function, and reduce the occurrence of complications.
3. The slowly-digested Pueraria montana var. thomsonii starch prepared by the preparation method of the present disclosure has few toxic and side effects, and exhibits a strong effect of preventing and/or improving non-therapeutic intervention of a blood glucose level.

### BRIEF DESCRIPTION OF THE DRAWINGS

To describe the technical solutions in the embodiments of the present disclosure clearly, the accompanying drawings required for describing the embodiments are briefly introduced below. Apparently, the accompanying drawings in the following description show merely some embodiments of the present disclosure, and those of ordinary skill in the art may still derive other drawings from these accompanying drawings without creative efforts.

FIG. 1 shows the comparison of RDS, SDS, and RS contents in Pueraria montana var. thomsonii starches obtained in Examples 1 to 3 of the present disclosure; and

FIG. 2 shows the comparison of RDS, SDS, and RS contents in commercially-available Pueraria montana var. thomsonii starches 1 to 3 of specified brands.

### DETAILED DESCRIPTION OF THE EMBODIMENTS

The principles and features of the present disclosure will be described in conjunction with the following examples, and the examples given are used merely to explain the present disclosure, rather than limit the scope of the present disclosure.

Example 1 A preparation method of a slowly-digested Pueraria montana var. thomsonii starch was provided in this example, specifically including the following steps:
(1) Fresh Pueraria montana var. thomsonii, a medicinal and edible plant, was taken as a raw material, and the raw material was cleaned, peeled, and chopped; and 52° C. hot water was added at a weight 5 times a weight of the raw material, and a resulting mixture was fully crushed, sieved to remove crude fibers, and allowed to stand.
(2) A supernatant obtained after the standing in the step (1) was collected and heated to 55° C., α-amylase was added according to a weight ratio of 0.1%, and a resulting mixture was stirred to allow enzymatic hydrolysis for 3 h, heated to 95° C., and kept at this temperature for 10 min to inactivate the enzyme; and a pressure of an inactivated system obtained after the enzyme inactivation was reduced to 0.15 KPa, a resulting solution was concentrated and centrifuged, and a resulting precipitate was discarded to obtain a first Pueraria montana var. thomsonii concentrate composition.
(3) A precipitate obtained after the standing in the step (1) was collected, and repeatedly cleaned with drinking water and settled 3 times, and a final precipitate was collected to obtain a second Pueraria montana var. thomsonii starch composition.
(4) The first Pueraria montana var. thomsonii concentrate composition obtained in the step (2) and the second Pueraria montana var. thomsonii starch composition obtained in the step (3) were mixed according to a weight ratio of 0.5:100, and 40 parts of deionized water were added for dilution to obtain a homogeneous slurry.
(5) The homogeneous slurry obtained in the step (4) was dried, crushed, and sieved through an 80-mesh sieve to obtain the slowly-digested Pueraria montana var. thomsonii starch, where the slowly-digested Pueraria montana var. thomsonii starch included 25% of SDS and 18.5% of RS.

Example 2 A preparation method of a slowly-digested Pueraria montana var. thomsonii starch was provided in this example, specifically including the following steps:
(1) Fresh Pueraria montana var. thomsonii, a medicinal and edible plant, was taken as a raw material, and the raw material was cleaned, peeled, and chopped; and 53° C. hot water was added at a weight 6 times a weight of the raw material, and a resulting mixture was fully crushed, sieved to remove crude fibers, and allowed to stand.
(2) A supernatant obtained after the standing in the step (1) was collected and heated to 55° C., α-amylase was added according to a weight ratio of 0.3%, and a resulting mixture was stirred to allow enzymatic hydrolysis for 3 h, heated to 95° C., and kept at this temperature for 10 min to inactivate the enzyme; and a pressure of an inactivated system obtained after the enzyme inactivation was reduced to 0.15 KPa, a resulting solution was concentrated and centrifuged, and a resulting precipitate was discarded to obtain a first Pueraria montana var. thomsonii concentrate composition.
(3) A precipitate obtained after the standing in the step (1) was collected, and repeatedly cleaned with drinking water and settled 3 times, and a final precipitate was collected to obtain a second Pueraria montana var. thomsonii starch composition.
(4) The first Pueraria montana var. thomsonii concentrate composition obtained in the step (2) and the second Pueraria montana var. thomsonii starch composition obtained in the step (3) were mixed according to a weight ratio of 2.5:100, and 50 parts of deionized water were added for dilution to obtain a homogeneous slurry.
(5) The homogeneous slurry obtained in the step (4) was dried, crushed, and sieved through an 80-mesh sieve to obtain the slowly-digested Pueraria montana var. thomsonii starch, where the slowly-digested Pueraria montana var. thomsonii starch included 17% of SDS and 14.6% of RS.

Example 3 A preparation method of a slowly-digested Pueraria montana var. thomsonii starch was provided in this example, specifically including the following steps:
(1) Fresh Pueraria montana var. thomsonii, a medicinal and edible plant, was taken as a raw material, and the raw material was cleaned, peeled, and chopped; and 55° C. hot water was added at a weight 8 times a weight of the raw material, and a resulting mixture was fully crushed, sieved to remove crude fibers, and allowed to stand.
(2) A supernatant obtained after the standing in the step (1) was collected and heated to 55° C., α-amylase was added according to a weight ratio of 0.6%, and a resulting mixture was stirred to allow enzymatic hydrolysis for 3 h, heated to 95° C., and kept at this temperature for 10 min to inactivate the enzyme; and a pressure of an inactivated system obtained after the enzyme inactivation was reduced to 0.15 KPa, a resulting solution was concentrated and centrifuged, and a resulting precipitate was discarded to obtain a first Pueraria montana var. thomsonii concentrate composition.
(3) A precipitate obtained after the standing in the step (1) was collected, and repeatedly cleaned with drinking water and settled 3 times, and a final precipitate was collected to obtain a second Pueraria montana var. thomsonii starch composition.
(4) The first Pueraria montana var. thomsonii concentrate composition obtained in the step (2) and the second Pueraria montana var. thomsonii starch composition obtained in the step (3) were mixed according to a weight ratio of 5:100, and 60 parts of deionized water were added for dilution to obtain a homogeneous slurry.
(5) The homogeneous slurry obtained in the step (4) was dried, crushed, and sieved through an 80-mesh sieve to obtain the slowly-digested Pueraria montana var. thomsonii starch, where the slowly-digested Pueraria montana var. thomsonii starch included 10% of SDS and 15.4% of RS.

Comparative Example 1: A commercially-available Pueraria montana var. thomsonii starch 1 of a specified brand, a commercially-available Pueraria montana var. thomsonii starch 2 of a specified brand, and a commercially-available Pueraria montana var. thomsonii starch 3 of a specified brand were purchased from the market, and RDS, SDS, and RS contents in the three Pueraria montana var. thomsonii starches were determined.

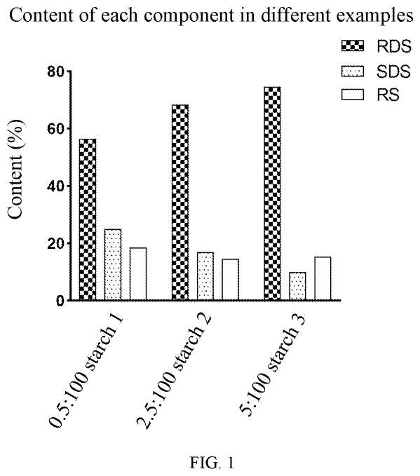

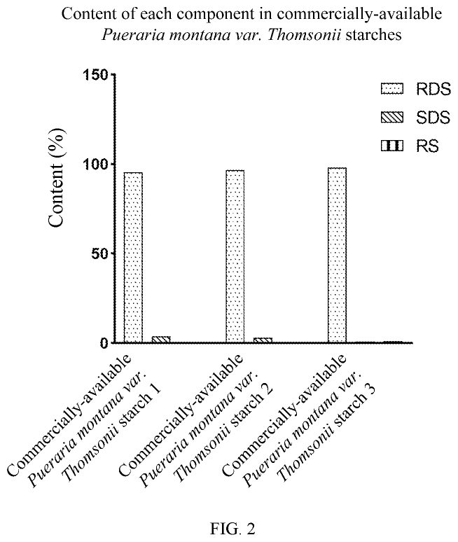

RDS, SDS, and RS contents in the Pueraria montana var. thomsonii starches prepared in Examples 1 to 3 of the present disclosure and the commercially-available Pueraria montana var. thomsonii starches 1 to 3 of specific brands were detailed in Table 1, and as shown in FIG. 1 and FIG. 2, the preparation method in the present disclosure can significantly increase SDS and RS contents in the Pueraria montana var. thomsonii starch.

TABLE 1
SDS and RS contents in Pueraria montana var. thomsonii
starches obtained through different processes

| | Content (%) | | |
|---|---|---|---|
| Sample | RDS | SDS | RS |
| Commercially-available | 95.49% | 3.80% | 0.71% |
| Pueraria montana var. thomsonii | | | |
| starch 1 of a specified brand | | | |
| Commercially-available | 96.65% | 3.11% | 0.24% |
| Pueraria montana var. thomsonii | | | |
| starch 2 of a specified brand | | | |
| Commercially-available | 98.03% | 0.97% | 1.00% |
| Pueraria montana var. thomsonii | | | |
| starch 3 of a specified brand | | | |
| Example 1: slowly-digested | 56.5% | 25% | 18.5% |
| Pueraria montana var. thomsonii | | | |
| starch 1 with 0.5:100 | | | |
| Example 2: slowly-digested | 68.4% | 17% | 14.6% |
| Pueraria montana var. thomsonii | | | |
| starch 2 with 2.5:100 | | | |
| Example 3: slowly-digested | 74.6% | 10% | 15.4% |
| Pueraria montana var. thomsonii | | | |
| starch 3 with 5:100 | | | |

The above are merely preferred specific implementations of the present disclosure, but the protection scope of the present disclosure is not limited thereto. Any modification or replacement easily conceived by those skilled in the art within the technical scope of the present disclosure should fall within the protection scope of the present disclosure.

What is claimed is:

1. A preparation method of a slowly-digested Pueraria montana var. thomsonii starch, comprising the following steps:
(1) taking fresh Pueraria montana var. thomsonii, a medicinal and edible plant, as a raw material, and cleaning, peeling, and chopping the raw material; and adding 52° C. to 55° C. hot water at a weight 5 to 8 times a weight of the raw material, fully crushing, sieving to remove crude fibers to obtain a resulting slurry, and allowing the resulting slurry to stand;
(2) collecting a supernatant obtained after the standing in the step (1), heating the supernatant to 52° C. to 55° C., adding α-amylase according to a weight ratio of 0.1% to 0.6% to obtain a resulting mixture, and stirring the resulting mixture to allow an enzymatic hydrolysis for 1 h to 3 h to obtain a resulting reaction system; heating the resulting reaction system to a temperature of 95° C. and holding the temperature for 5 min to 10 min to inactivate the enzyme to obtain an inactivated system, and reducing a pressure of the inactivated system obtained after the enzyme inactivation to 0.1 KPa to 0.15 KPa to obtain a resulting solution; and concentrating and centrifuging the resulting solution to obtain a resulting precipitate, and discarding the resulting precipitate to obtain a first Pueraria montana var. thomsonii concentrate composition;
(3) collecting a precipitate obtained after the standing in the step (1), repeatedly cleaning and settling the precipitate with drinking water 2 to 3 times, and collecting a final precipitate to obtain a second Pueraria montana var. thomsonii starch composition;
(4) mixing the first Pueraria montana var. thomsonii concentrate composition obtained in the step (2) and the second Pueraria montana var. thomsonii starch composition obtained in the step (3) according to a weight ratio of (0.5-5):100, and adding 40 to 60 parts of deionized water for dilution to obtain a homogeneous slurry; and
(5) drying and crushing the homogeneous slurry obtained in the step (4) to obtain a crushed material, and sieving the crushed material through an 80-mesh sieve to obtain the slowly-digested Pueraria montana var. thomsonii starch.

2. The preparation method of the slowly-digested Pueraria montana var. thomsonii starch according to claim 1, wherein the slowly-digested Pueraria montana var. thomsonii starch comprises 10% to 25% of slowly-digested starch (SDS) and 8% to 20% of resistant starch (RS).

3. The preparation method of the slowly-digested Pueraria montana var. thomsonii starch according to claim 1, wherein in the step (4), the first Pueraria montana var. thomsonii concentrate composition and the second Pueraria montana var. thomsonii starch composition are mixed according to a weight ratio of 5:100.

4. A use method of the slowly-digested Pueraria montana var. thomsonii starch prepared by the preparation method according to claim 1 as a major raw material in a development and production of a low-glycemic index (GI) cooked wheaten food, pastry, and solid beverage.

5. A use method of the slowly-digested Pueraria montana var. thomsonii starch prepared by the preparation method according to claim 1 in a preparation of a product for a non-therapeutic intervention of a blood glucose level.

6. The use method of the slowly-digested Pueraria montana var. thomsonii starch according to claim 5, wherein the non-therapeutic intervention of the blood glucose level is manifested as at least one selected from the group consisting of the following conditions (a) to (d):
(a) regulating the blood glucose level;
(b) extending a time required to reach a blood glucose peak;
(c) lowering the blood glucose level and improving a glucose tolerance; and
(d) improving a pancreatic islet function.
